(19) United States
(12) Patent Application Publication
(10) Pub. No.: US 2018/0342315 A1
(43) Pub. Date: Nov. 29, 2018

(54) METHOD FOR 3D IMAGING OF MECHANICAL ASSEMBLIES TRANSPLANTED INTO MAMMALIAN SUBJECTS

(71) Applicant: HALIFAX BIOMEDICAL INC., Mabou (CA)

(72) Inventors: Johan Erik Giphart, Mabou (CA); Yann Gagnon, Mabou (CA)

(73) Assignees: HALIFAX BIOMEDICAL INC., Mabou, NS (CA); HALIFAX BIOMEDICAL INC., Mabou, NS (CA)

(21) Appl. No.: 15/755,936

(22) PCT Filed: Aug. 31, 2016
(86) PCT No.: PCT/CA2016/051025

§ 371 Date: Feb. 27, 2018

Related U.S. Application Data
(60) Provisional application No. 62/212,265, filed on Aug. 31, 2015.

(51) Int. Cl. G16H 30/40; G06T 7/50

(52) U.S. Cl. CPC G16H 30/40; G06T 7/50

(57) ABSTRACT

A medical imaging method is described for establishing implant configuration by measuring the positions and orientations of its components constrained by a defined kinematic relationship between them using stereo radiography images. The method allows the determination of a patient-specific implant configuration and enables accurate measurements of the pose of implant despite significant occlusions. The measurements obtained with this method can be used to determine the relative movement of an implant component relative to other components, bone or other landmark.

### TECHNICAL FIELD

The present invention relates to the field of medical imaging and, in particular, to 3D medical imaging of implanted joint replacement components.

### BACKGROUND

Osteoarthritis (OA) is the most common cause of arthritis, and is one of the leading causes of disability. OA significantly affects an individual's ability to work and decreases their quality of life. OA is a degenerative joint disease where the cartilage of a joint, such as the knee or hip, is compromised resulting in swelling, stiffness and pain. Joint replacement surgery using an orthopedic implant is the typical course of treatment when pain and/or loss of function become severe.

In the United States, the cost of joint replacement surgery has been reported to total nearly 50 billion USD in 2009, surpassing 1 million hip and knee replacements annually in recent years. The continued growth of arthroplasty procedures will also increase the burden of revision surgeries due to prosthesis problems, including implant loosening and assembly failures.

Stereo radiography is a technique that uses two x-ray systems with intersecting beams and taking two x-ray images simultaneously of an object placed in the beam intersection. Stereo radiography has traditionally been used to accurately measure migration which is the micromotion of an implant over time relative to bone. Accuracy and precision of 0.1 mm can be achieved using stereo radiography. Excessive migration within the first year or two has been demonstrated to be able to predict the need for revision surgery due to implant loosening as much as 10 years later and well before symptoms occur. This enables stereo radiography to detect problems with specific implants earlier and with fewer patients than other methods.

The assessment and monitoring of implants using stereo radiography methods such as radio stereometric analysis (RSA) requires an imaging setup capable of high measurement accuracy and precision. In addition to knowing the imaging configuration to a high degree of accuracy and precision, 3D computer models of the implant being measured are also necessary for the analysis. Current analysis methods assume an implant is made of one component or a fixed and known configuration of components, or otherwise each component must be measured independently. However, in the case where an implant is an assembly consisting of multiple components, the precise configuration of the components making up the implant assembly may not be known and even be patient-specific due to tolerance stack-up within the assembly.

An assessment may be further complicated by a limited field of view or occlusion of part of the assembly caused by radio-opaque components of the assembly itself or other implant components, such as a radiopaque cup occluding the head on the femoral stem of a hip replacement implant. In such cases, it may be impossible to accurately localize specific components of the assembly in the traditional manner. That is, there may not be enough image information available to resolve all 6 degrees of freedom describing the pose, comprised of the position (x-coordinate, y-coordinate, z-coordinate) and orientation (i.e., rotations about the x-axis, y-axis, and z-axis) of the component. The loss of accuracy and precision because of this missing information can be prohibitive in assessing and monitoring implants using stereo radiography.

### SUMMARY

The exemplary embodiments of the present disclosure relate to methods for measuring the 3D configuration of an orthopaedic implant assembly, its 3D position and orientation relative to bone as well as relative to another implant or implant component using stereo radiography.

One exemplary embodiment relates to a method for measuring implant location in a patient, wherein the method comprises: (a) 3D computer models of the components which make up an orthopedic implant assembly, (b) defined kinematic relationships of the implant assembly's components, wherein a principal component is defined and the position and orientation of all other secondary components are described relative to the principal component or the preceding component in the kinematic chain, (c) the acquisition of stereo radiographic imaging data, and (d) accurate measurement of the configuration of the implant's assembly as well as position and orientation of the implant using the constraints of the kinematic relationships of its components. According to some exemplary embodiments, the method further comprises: (e) using the assembly configuration and 3D pose obtained from at least two time points to measure changes in assembly configuration and/or pose relative to bone or to another implant or implant component.

The method disclosed herein may use location(s) of the clearly visible component(s) of an implant assembly, combined with knowledge of the kinematic relationship between the implant components and the limited information from the partially occluded components, to accurately determine the configuration of the assembly and 3D location of the occluded component(s) within the patient wherein the implant assembly is installed.

### BRIEF DESCRIPTION OF THE DRAWINGS

These and other features of the invention will become more apparent in the following detailed description in which reference is made to the appended drawings.

FIG. 1 is a schematic illustration of a stereo radiography system in a 60 degree inter-beam configuration that may be used in an exemplary method, according to an embodiment of the present disclosure.

FIG. 2 is a schematic illustration of an image registration and creation of a common reference frame (coordinate system) based on the sets of markers provided by the reference box of the exemplary dynamic stereo radiography system shown in FIG. 1;

FIG. 3 is a display illustrating implant tracking between a three-dimensional model and a pair of radiographic images to optimize position and orientation for each component of the implant assembly, according to an exemplary embodiment of the present disclosure;

FIG. 4 is a flowchart illustrating the workflow leading to and including the optimization of the position and pose of the components making up an implant assembly;

FIG. 5 is a schematic illustration of an exemplary prismatic kinematic between the femoral stem (principal component) and femoral head (secondary component), according to an embodiment of the exemplary methods disclosed herein; and

FIG. 6 is a is a schematic illustration showing a representation of a wear measurement in an acetabular cup liner using the configuration and pose of the components of an implant assembly, according to an exemplary method disclosed herein.

### DETAILED DESCRIPTION OF THE INVENTION

Imaging-based measurements of orthopaedic implants in vivo with stereoradiography enable the assessment and monitoring of implant loosening and provide data predictive of revision surgery and patient outcome.

The embodiments of the present disclosure describe methods based on stereo radiography that allow the configuration of the individual components of an implant assembly to be quantitatively determined in 3D. Specifically, the embodiments of the present disclosure include adding additional degrees of freedom to the pose optimization of an implant assembly per the kinematic relationship between the components resulting in the implant assembly's configuration, position and orientation.

Some exemplary embodiments of the present disclosure pertain to methods in which the position and orientation of the implant assembly's components are used to measure metrics of interest such as settling of assembly components onto each other, bedding in, creep, and wear in implants with liners or spacers, migration of the implant within the bone into which it has been installed.

For purposes of illustration, the devices and methods of the invention are described below with reference to the in vivo measurement of the femoral components of a human hip implant. However, as will be appreciated by those skilled in the art, the methods can be employed with other types of implant assemblies for example knee implants, shoulder implants, other joints, in vitro or in situ, and for any mammal.

The exemplary embodiments of the present disclosure relate to the 3D determination of the configuration of an implant assembly installed into a mammalian subject, as well as the position and orientation of the implant assembly's components. Specifically, 3D computer models of the implant assembly's components are obtained and their assembly and pose determined based on a stereo pair of radiographic images of a patient's implant. By comparing measured positions and orientations at multiple time points, metrics of interest such as migration, creep and wear, and component settling can be measured. A person skilled in the art will also recognize that a series of radiographic images can be obtained in a dynamic manner or a series of progressive static radiographic images, with or without a prescribed voluntary motion performed by the patient. A person skilled in the art will also recognize that the methods described herein may also be used in single plane x-ray images at a likely expense of accuracy and precision.

### Stereo Radiography Imaging

Persons of ordinary skill in this art will recognize that there are a variety of stereo-radiography techniques that may be used to obtain the radiographic images of the implant assembly. For example, biplane or dual-plane fluoroscopy of radiostereometric analysis (RSA). Some exemplary embodiments of the present disclosure relate to a stereo-radiographic imaging method for obtaining three-dimensional measurements of an implant's position and orientation within a target region of a patient's anatomy that comprises capturing stereo x-ray exposures of a patient who is upright or lying on a table. According to further embodiments, as is readily understood by those skilled in the art, weights, rubber bands, and the like, can be used to load the joint which contains the implant.

Persons of skill in the art will recognize that a variety of methods may be used to obtain the 3D position and orientation of the implant assembly's components from the radiographic images. Without limiting the foregoing, reference objects may be included in the field of view to allow the calculation of the imaging configuration. Moreover, the image information used to calculate the 3D position and orientation may be based on the use of edge detection of the radiographic images, gradient information obtained from the image, feature recognition and extraction or digitally reconstructed radiography combined with image matching.

### Measurement of the Position and Orientation of an Implant Assembly's Components

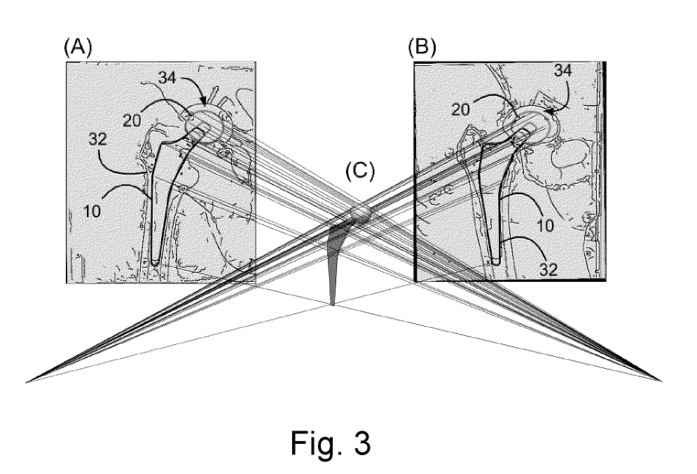

The three-dimensional measurement of the position and orientation of the implant assembly's components consists of establishing a geometric relation between the implant's representation in the stereo radiographic images and a 3D computer model of the implant assembly's components. According to some exemplary embodiments of the present disclosure, methods for the 3D measurement involve fitting the projection of the 3D computer model to edge or gradient data of the implant assembly's components visible in the radiographic images. In this way, the position and orientation of the 3D computer model of the implant assembly's components are derived from the radiographic images thereby resolving the configuration of the implant assembly (FIG. 3).

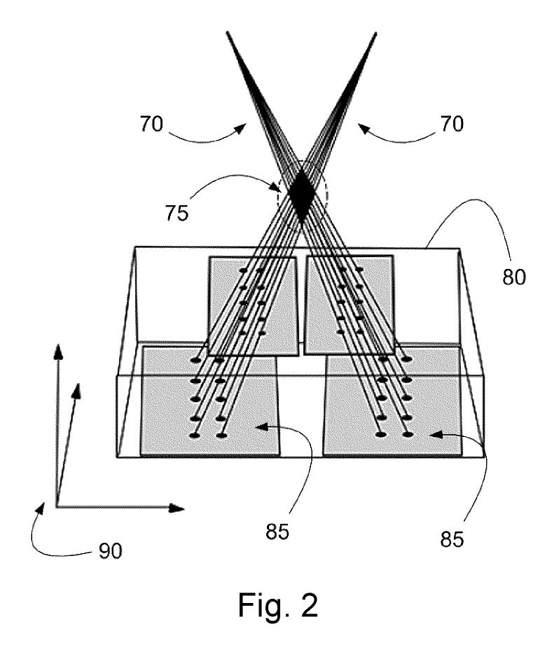

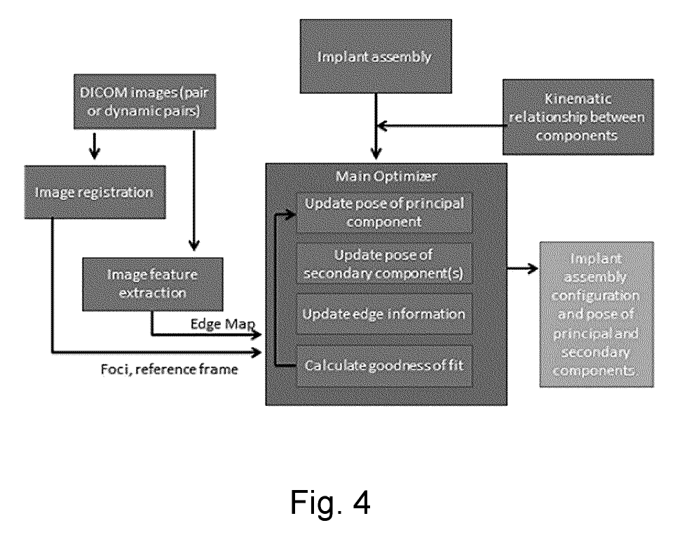

Image registration is performed either through known information about the imaging configuration or by determining the imaging configuration using the radiographic images. According to an exemplary embodiment, this involves determining x-ray foci positions from the stereo radiographic images and consolidating all image information into a common reference frame. According to an exemplary embodiment of the present disclosure, a registration element exemplified by a reference box (FIG. 2) is positioned between the patient and the detector panels. The registration element has a series of fiducial and control beads that provide reference markers from which x-ray foci can be calculated and all image information can be consolidated in a common reference frame (FIG. 4).

Image feature extraction, according to embodiments of the present disclosure, includes filtering of the images for improved image quality, the robust detection of edges in the images, and the creation of component-specific edge maps.

The 3D computer models of the components of the implant assembly can be obtained using a variety of methods known to those skilled in the art. According to embodiments of the present disclosure, the 3D computer models can be generated from CAD software. According to other embodiments, the 3D computer model can be generated by optical scanning. According to other embodiments, the 3D computer model can be represented by a parametrized geometric model. According to other embodiments, the 3D computer model can be generated from a CT or MRI scan.

It is to be noted that the 3D computer models of the components of the implant assembly are defined separately. A principal component, from which the position and orientation is assigned to the entire assembly, is chosen from the assembly and from which the kinematic chain of secondary components is defined. Further, kinematic relationships between each of the secondary components and the principal component are defined, thereby constraining the possible configurations of the assembly and reducing the degrees of freedom needed to solve the configuration of the assembly. It should be noted that for the special case of a component being independent from all other components, no secondary components are linked. According to another embodiment of the present disclosure, more than one kinematic chain can be defined and measured concurrently.

The main optimizer involves fitting the general three-dimensional position and orientation of the assembly and configuration of the components to establish a best-fit (FIG. 4). These iterations involve optimizing the absolute position and orientation of the principal component of the implant assembly, along with the relative positions of the secondary components as allowed by the kinematic relationships of the implant assembly. The steps in the main optimizer are repeated for each image pair to obtain the optimized positions and orientations; in absolute terms for the principal component and in relative terms for each secondary component of the implant assembly. For each secondary component, the resulting output can be converted to absolute positions and orientations (FIG. 4).

Another exemplary embodiment of the present disclosure pertains to updating of the edge data from the edge map at each iteration based on goodness of fit with the projected 3D computer models.

### Measuring Changes in Configuration, Pose and Relative Pose

According to exemplary embodiments of the present disclosure, the optimized 3D computer model of the components of the implant assembly provides the basis for accurate quantitative measurement of metrics of interest in the assessment or monitoring of an orthopedic implant. In particular, migration of the implant assembly relative to bone as in traditional stereo radiography can be determined. When varying loading conditions, changes in assembly configuration suggest a loosening of one or more components within the assembly. According to particular embodiments, the change in the relative three-dimensional position and orientation of the femoral head relative to the acetabular cup between two time points can be used to calculate wear of the acetabular cup's liner.

### EXAMPLES

### Example 1

### Imaging Apparatus

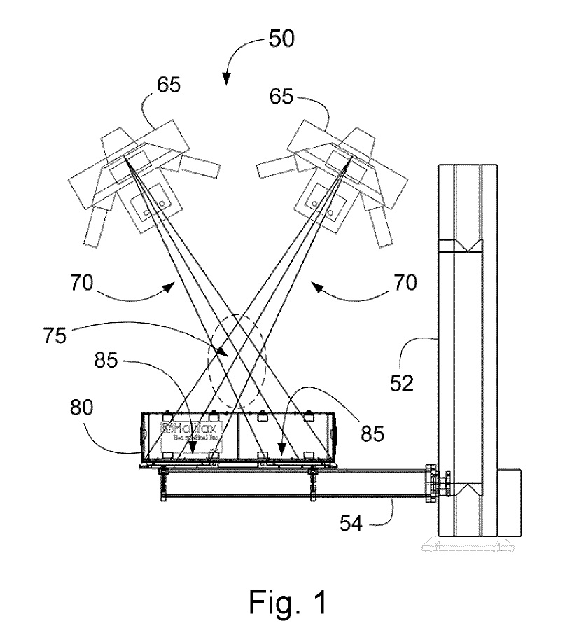

A stereo orthopaedic radiography system 50 (Halifax Imaging Suite; Halifax Biomedical Inc., Mabou, NS, Canada) was used. The stereo orthopaedic radiography system 50 comprised two radiography systems 65 exposing simultaneously to obtain stereo radiographic images (FIG. 1). Each radiography system 65 comprised an x-ray source (RAD-92 Sapphire X-Ray Tube; Varian Medical Systems, Palo Alto, Calif., USA), a generator (Hydravision SHF635RF DR X-Ray Generator, SEDECAL USA Inc., Buffalo Grove, Ill., USA), an x-ray detector panel 85, a digital imaging system (CDXI 50RF, Canon USA Inc., Melville, N.Y., USA), and a computer system to link the components together, to retrieve the imaging data, and to reconstruct the imaging data. The two x-ray imaging systems 65 are positioned at an angle to each other such that their x-ray beams 70 overlap in part to create a 3D viewing volume 75.

A 60-degree reference box 80 (SR Reference Box; Halifax Biomedical Inc., Mabou, NS, Canada) was placed into the image field of both systems 65 (FIGS. 1, 2). The reference box 80 was constructed from carbon fiber to insure rigidity, to resist deformations resulting from temperature fluctuations during operation, and for its radiolucency. The reference box 80 housed two digital detector plates 85 in the bottom (away from the patient and x-ray source) in a uniplanar configuration, immediately behind a fiducial plane which contained a series of equidistantly spaced radio opaque tantalum beads. The top of the box 80 formed the control plane which contained radio-opaque tantalum beads also. The fiducial beads allowed the captured images to be transformed to a common reference frame, while the control beads allowed the calculation of the foci (i.e., the x-ray sources) locations to enable the analysis. The images were captured on two digital detector plates 85 (CDXI 50RF, Canon USA Inc., Melville, N.Y., USA) as greyscale images with relative intensity values in standard medical DICOM format. The overlap of the two radiography systems' fields of view made up the 3D viewing volume 75 (FIG. 2). The registration element has a series of fiducial and control beads that provide reference markers from which x-ray foci can be calculated and all image information can be consolidated in a common reference frame 90. The reference box 80 is securely mounted onto a beam 54 that is pivotably engaged with a vertical support column 52 whereby the beam 54 can be controllably raised upward and downward and additionally controllably rotated on the vertical support column 52 (FIG. 1).

### Image Data Acquisition

Images were acquired with the patients in supine and standing positions. For each image, the patients were positioned and instructed by a technologist on how to hold the position. Each of the image pairs were reviewed by the technologist to ensure image quality and the regions of interest were captured. The images were then transferred using tele-radiology technology to the image analysis center for analysis.

Definition of Implant Assembly and Kinematic Relationship

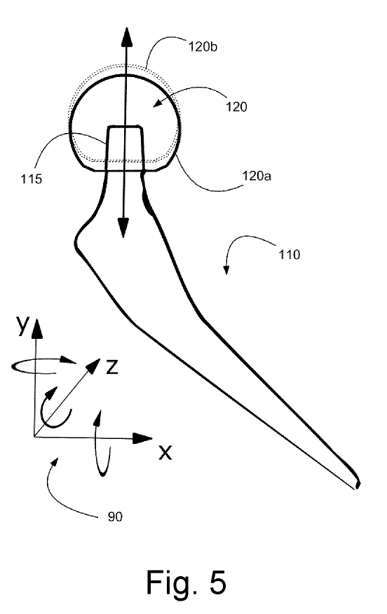

An orthopaedic implant designed for total hip replacement installed into a patient was imaged post-operatively as described above. The components making up the hip implant are a femoral stem 10 and femoral head 20 installed into their femur 32, and an acetabular cup and a polyethylene liner (not shown) installed into the socket 34 of their pelvis (FIG. 3). A 3D computer model (C) of these components was calculated from the two radiographic images (A), (B) concurrently captured by the two radiography systems 65 (FIG. 3) following the steps outlined in FIG. 4. In this example, the femoral head comprised ceramic material which is relatively radio-lucent while the acetabular cup was made from tantalum which is radio-opaque, thereby rendering the femoral head significantly occluded in one or both radiographic images (A), (B) (FIG. 3). The degree and location of occlusion depended on patient positioning and could not be predicted. The purpose for imaging was to measure cup liner wear which is defined for this purpose as the penetration of the head into the cup, in the proximal direction, at multiple time points. The degree of occlusion in most image sequences prohibited this calculation using the standard techniques known in the art. However, the femoral stem was visible in its entirety in all image sequences. Therefore, the femoral stem was chosen as a principal component of the implant assembly with the femoral head as the secondary component. The kinematic relationship between the femoral stem and the femoral head was defined, as prismatic coupling with the axis of symmetry of the neck of the stem and the axis of symmetry of the head set to be collinear (FIG. 5). A reasonable starting location was set for these two components. Thus, the assembly of the femoral component of the hip implant was described as a 7 degrees of freedom system with the pose of the femoral stem described by 6 degrees of freedom (three translations and three rotations) and the position of the femoral head onto the stem as the seventh degree of freedom. The seventh degree of freedom was relative to the femoral stem and described the translation of the femoral head along the collinear symmetry axes, from the initial position. The acetabular cup was clearly visible in all images and was defined as an independent component of the implant and described by all 6 degrees of freedom (FIG. 5). The polyethylene liner of the hip implant was not visible in the x-rays (FIG. 3(A), (B)) and could not be measured.

### Determination of Imaging Configuration

The radiographic images were loaded onto a computer system for calculation of the parameters that described the detailed configuration of the imaging system. The fiducial beads in the reference box were located in the images and their locations tabulated. Based on the known locations of these beads, a projective transformation was calculated that matched the bead locations to the tabulated locations from the images following the process steps outlined in FIG. 4. The control beads of the reference box were located in the images and their locations tabulated. Based on the known locations of the fiducial beads and the control beads, the locations of the two foci were calculated.

### Extraction of Image Features

The radiographic images were filtered using a Canny edge detection filter. Using a graphical user interface, a trained user selected all the edges belonging to the femoral stem, head and acetabular cup separately. An initial position and orientation for the femoral stem (with the coupled head) and cup were set by the user, also using a graphical user interface.

### Determination of Implant Assembly Configuration and Component Pose

The location of the foci and the parameters describing the projective transform were used to calculate the projected contours onto the fiducial plane for any given position and orientation of the components making up the implant. An objective function was made available to the optimizer which calculated a goodness-of-fit score between the projected contours and user-selected component-specific edge maps, given the pose of the stem, the relative translation of the head along the symmetry axis and the pose of the cup. The goodness of fit score was based on a sum of squared distance metric and was calculated separately for the femoral stem and femoral cup.

The optimizer used the objective function to find the configuration of the implant assembly which provided the best fit to the radiographic images, within a predefined search space. In this example, the optimizer first used Particle Swarm Optimization as a global optimization method. A second round of optimization attempted to further increase the goodness-of-fit with a local, gradient-based optimizer. The initial position of the particles was uniformly distributed along the predefined search space and centered on the user initialized estimates. The optimizer returned the final pose of the stem 110, neck 115 of the stem 110, and translation of the femoral 120a, 120b relative to the stem 110 along the axis of symmetry 90, and, the pose of the cup (FIG. 5).

### Calculation of Cup Liner Wear

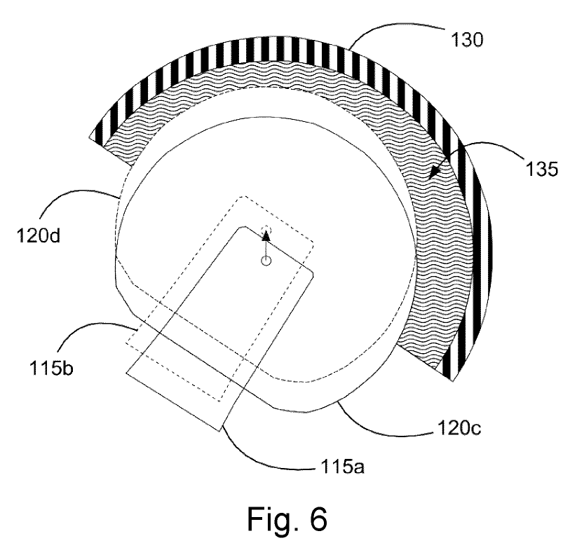

Cup liner wear was defined as proximal penetration of the head into the cup. With the implant configuration determined by the pose of the femoral neck 115 and the relative position of the femoral head 120 to the femoral stem 115, the absolute pose of the head was calculated for each time point, i.e., “120c” at 1 year and “120d” at 2 years (FIG. 6). To calculate the displacement of the head relative to the cup, the pose of the cup 120d at 2 years was transformed to be coincide with the pose of the cup at 1 year 120c; thus using the 1-year pose as the reference. The same transform was applied to the head's pose at 2 years. In this way, a displacement vector could be determined describing the motion of the head relative to the cup between the two time points. The component of this displacement generally aligned with the proximal anatomical direction and was reported was cup liner wear.

1. A method for quantitatively measuring the individual position and orientation of components making up an implant assembly in a patient, the method comprising:
a) capturing a series of stereo radiographic images of a target region;
b) measuring the position and orientation of each components of an implant assembly, calculated based on the radiographic images of the series of images; and
c) measuring a change in the relative three-dimensional position and orientation of each component of the implant assembly, wherein the measurements between time points can be used to calculate the relative motion between one component to another, or alternatively, between one component and a selected landmark.

2. The method according to claim 1, further comprising:
d) calculating the three-dimensional position and orientation of each component of an implant assembly, wherein a defined kinematic relationship restricts the relative movement of one or more secondary components to a principal component.

3. The method according to claim 1, wherein calculation of the absolute and relative three-dimensional position and orientation for each component of an implant assembly in step (b) is by iterative optimization based on the radiographic images for each frame of the series of images.

4. A method for assessing metrics of interest relating to the assessment or monitoring of an orthopedic implant:
a) capturing a series of stereo radiographic images of a target region of the patient containing the implant;
b) defining kinematics relationship of the components of the implant, wherein a principal component is defined and with the motion of all other secondary components being defined with respect to the principal component, wherein the degrees of freedom of this relative motion is reduced by constraints which are relevant to the implant's design.
c) measuring a change in the relative three-dimensional position and orientation of each component in the implant assembly or other landmark; and
d) using the measured change in the three-dimensional model to calculate metrics of interest such as acetabula cup liner wear.

5. The method according to claim 4, wherein calculation of the relative three-dimensional position and orientation for each component of the implant assembly in step (b) is by iterative optimization based on the radiographic images for each frame of the series of images.

6. A radiographic imaging method for calculating the position and orientation of the components of an implant assembly, the method comprising:
a) capturing a series of radiographic images of the target region, the radiographic images comprising a pair of images taken at an angle of each other to capture images within a viewing volume;
b) calculating foci and edge data of the components of the implant assembly captured in a radiographic image in the series and consolidating the data to a common reference frame;
c) determining the absolute and relative three-dimensional position and orientation of the components of an implant assembly;
d) iteratively manipulating the general three-dimensional position and orientation of the components of an implant assembly against the data in the common reference frame to achieve a best-fit three-dimensional position and orientation for each component in the radiographic image; and
e) repeating steps b to d for each image pair of a series;

7. The method according to claim 6, wherein a kinematic relationship between the components of the implant assembly is used to restrict the relative motion of secondary components to a principal component.

8. The method according to claim 6, wherein the 3D computer models of the components of an implant assembly are digital CAD models.

9. The method according to claim 6, wherein the 3D computer models of the components of an implant assembly are reconstructed from an optical scanner.
